(19) United States
(12) Patent Application Publication
(10) Pub. No.: US 2008/0292571 A1
(43) Pub. Date: Nov. 27, 2008

(54) Deodorant composition with monocyclic, unsaturated sequiterpene alcohols and glyceryl monoalkyl ethers as synergistic active deodorant agents

(76) Inventor: Joo W. Kim, Mississauga (CA)

Correspondence Address: The Halvorson Law Firm, 1757 E. Baseline Rd., Ste 130, Gilbert, AZ, 85233, US

(21) Appl. No.: 11/805,957

(22) Filed: May 24, 2007

(51) Int. Cl. A61K 8/34; A61K 8/19

(52) U.S. Cl. 424/66

(57) ABSTRACT

A clear deodorant stick composition consisting essentially of from 45 to about 75% of glycerin as solvent for soap gelling agent; and from 0.5 to about 2% alkali metal bicarbonate and from 0.5 to about 2.5% nonionic surfactants as clarity enhancer; and from 0.1 to about 0.5% natural alpha Bisabolol and from 0.3 to about 1% glyceryl monoalkyl ether as active deodorizing agents.

### FIELD OF THE INVENTION

The present invention relates to clear stick deodorant compositions. More particularly, compositions that include glycerin and water as solvent, soap gelling agent, alkali metal bicarbonate and ethylhexylglycerin as clarity enhancer, non-ionic surfactants and fatty alcohol as clarity enhancer and crystallization inhibitor, ethylhexylglycerin and alpha-Bisabolol as deodorizing active agents, and fragrance as malodor-masking agents.

### BACKGROUND

Personal care deodorants are typically intended for topical application to cover odors caused, at least in part, by perspiration. Typically, these deodorants are topically applied to an area of the skin, such as the underarm, and are in the form of soft deodorant gels or pressed powder sticks. In the gel form of these deodorants, it is common to find at least a gelling agent, a solvent, and a deodorant active material, such as perfume or other fragrances. Additional ingredients may be added for special benefits and/or effects.

Fatty acid soaps are commonly used as gelling agents in topical deodorant gels, such as alkali metal salts of naturally occurring fatty acids. The gelling agent is generally used in combination with a highly polar alcohol solvent to provide aesthetics such as clarity, ease of application, cool and refreshing feel on application, lack of powdery residue, and dry feel. Monohydric and dihydric alcohols, especially propylene glycol and dipropylene glycol, are often used for this purpose. Although these glycol containing deodorant gels are quite popular and commonly used, many are also harsh to the skin and can cause excessive skin irritation after topical application.

As deodorant active ingredients, triclosan is most popular and effective anti-microbial substances. However this substance contains organically bound halogen and is therefore not very environmentally compatible, since the formation of highly toxic dioxins during production or thermal decomposition cannot be ruled out. Therefore Chappell et al. incorporated glyceryl monolaurate in their deodorant composition (U.S. Pat. Nos. 5,256,405 and 5,260,053). But the antibacterial action of this substance is only a consideration when the pH reaches the range of from 6.0 to 7.0 in the axially vault. The pH of most soap gelling deodorant sticks is in the range from 9.0 to 10.0 and activity would not be observed until normal skin pH is restored.

U.S. Pat. No. 5,516,510 prepared an ethanol base deodorant that includes ethylhexylglycerin as active ingredient and they found 0.3% by weight ethylhexylglycerin is just as effective as 0.1% by weight triclosan. U.S. Pat. No. 5,736,574 reported an anti-microbial synergic effect of ethylhexylglycerin with N-n-octanoylglycin but this patent also report the anti-microbial activity against odor-causing gram positive bacteria of ethylhexylglycerin is very low.

However, there is a constant and continuing need for new deodorant formulations that have improved anti-microbial activity.

### SUMMARY OF INVENTION

It is therefore an object of the present invention to provide a gel deodorant composition which is milder to the skin and which causes little or no skin irritation.

It is another object of the present to provide such a composition having improved dry feel and other cosmetic benefits such as ease of application, emolliency, fragrance longevity, and/or reduced visible residue.

It is yet another object of the present invention to provide a combinatory anti-microbial mixture active against gram-negative bacteria comprising at least one glyceryl monoalkyl ether and an anti-microbially synergistically effective amount of at least one monocyclic, unsaturated sesquiterpene alcohol.

It is still yet another object of the present invention to provide the anti-microbial active above wherein the at least one glyceryl monoalkyl ether is ethylhexylglycerin.

It is a further object of the present invention to provide the anti-microbial active above wherein the at least one monocyclic, unsaturated sesquiterpene alcohol is alpha-bisabolol.

It is yet a further object of the present invention to provide the anti-microbial active above for use in pharmaceuticallcosmetic compositions.

It is still yet a further object of the present invention to provide a propylene glycol free anti-microbial composition for use in pharmaceutical/cosmetic compositions.

It is another object of the present invention to provide a clear, propylene glycol free, deodorant stick composition comprising a gelling agent, a solvent for the gelling agent and an effective amount of a deodorant active material comprising at least one glyceryl monoalkyl ether and an anti-microbially synergistically effective amount of at least one monocyclic, unsaturated sesquiterpene alcohol.

It is yet another object of the present invention to provide the above clear, propylene glycol free, deodorant stick composition wherein the glyceryl monoalkyl ether is ethylhexylglycerin.

It is still yet another object of the present invention to provide the above clear, propylene glycol free, deodorant stick composition wherein the monocyclic, unsaturated sesquiterpene alcohol is alpha-Bisabolol.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with any accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DETAILED DESCRIPTION OF PREFERRED EMBODIMENTS

The present invention is useful as a deodorant composition.

The composition, according to the present invention comprises at least a gelling agent, a gel solvent, and a deodorant active material. According to the present invention, the gelling agent, in the preferred embodiment, is a vegetable origin sodium fatty acid soap or sodium hydroxide and vegetable origin sodium fatty acid, such as sodium stearate, and most preferably Cl grade sodium stearate. The gelling agent is preferably present at about 5-10% by weight, with a range of about 5-8% weight percent being most preferred.

According to the present invention, the preferred gel solvent is a mixture of vegetable origin glycerin and water. The preferred ratio, by weight percent, of glycerin to water is generally 2/1 with the total weight percentage of this solvent mixture generally in the range of 85-93%.

To find out the substitute for triclosan, we tested natural extract like as lichen extract (usnic acid), ethylhexylglycerin, and natural alpha-Bisabolol. In case of natural extract, we have to use at least higher than 10% to get deodorant effect and there are some stability problems like as color change.

Ethylhexylglycerin is known as a deodorant active material with anti-microbial properties. Ethylhexylglycerin is a synthetic representative of the 1-alkyl glycerin ethers with a high degree of purity, with a 2-ethylhexyl group bound to the primary hydroxyl function of the glycerol molecule. The latter are linked at the C-3 atom via a phosphate group with amino-ethanol, choline or serine. Ethylhexylglycerin is a crystal-clear, colourless liquid with a slightly characteristic odour. It is only partly soluble in water (0.2%) and completely soluble in fats and organic solvents like alcohols, glycols and glycol ethers. Ethylhexylglycerin is rather stable, e.g. against hydrolysis and elevated temperatures and compatible with cosmetic ingredients. Ethylhexylglycerin is not a naturally occurring substance, but the glycerol ether structure occurs e.g. in batyl alcohol (octadecyl glycerol ether), which is found in shark liver oil. Other substances with a glycerin ether structure that occur naturally are the alkoxylipids, which are widely distributed in human and animal tissue.

Natural alpha-Bisabolol is an optically active monocyclic, unsaturated sesquiterpene alcohol containing a minimum of 95% as active (−)-alpha-bisabolol isomer, derived via the distillation of essential oils from Cananga ordorata, Citrus bigaradia, Lavandula spica, Matricaria chamomilla, Myoporum crassifolium, Myrocarpus fastigiatus, Myrocarpus frondosus and Vanillosmopsis erythropappa. Natural alpha-Bisabolol has anti-inflammatory, skin irritant reducing and anti-mycotic properties.

Alkali metal bicarbonates, such as sodium or potassium bicarbonate, have long been recognized for their deodorant properties, and have commonly been used as deodorants in refrigerators and the like. However, in the present invention, alkali metal bicarbonates are incorporated as clarifying agents in amounts ranging from about 0.5 to 3.0% by weight, preferably about 0.75 to about 2.0% by weight.

Hydrophilic and hydrophobic non-ionic surfactants that are incorporated in the formulation according to the present invention alkoxylated alcohols. Preferred hydrophilic alkoxylated alcohols are steareth-20, steareth-100 and preferred hydrophobic alkoxylated alcohols are oleth-2, laureth-4, isosteareth-2. The preferred HLB of this mixture in the range of around 16 to around 17 and total amount is in the range of around 0.5-3.0% by weight.

### Example 1

The effect of the sodium stearate amount was studied and the results are summarized in Tables 1 and 2, below. The compositions, which are set forth in Table 1, were prepared by first mixing deionized water and sodium bicarbonate, then adding glycerin and heating to 80-90° C. Subsequently, the steareth-100, sodium stearate, and stearyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 1

| | Concentration (wt. %) | | |
|---|---|---|---|
| Constituent | A | B | C |
| Glycerin | 60.00 | 60.00 | 60.00 |
| Deionized water | 30.85 | 30.35 | 29.85 |
| Sodium bicarbonate | 1.25 | 1.25 | 1.25 |
| Steareth-100 | 1.50 | 1.50 | 1.50 |
| Sodium Stearate | 4.50 | 5.00 | 5.50 |
| Stearyl Alcohol | 0.50 | 0.50 | 0.50 |
| Ethylhexylglycerin | 0.60 | 0.60 | 0.60 |
| Alpha Bisabolol (Natural) | 0.20 | 0.20 | 0.20 |
| Fragrance | q.s. | q.s. | q.s. |

TABLE 2

| Composition | Comments | 4° C. and 8 weeks |
|---|---|---|
| A | Clear | a lot of amorphous particles and crystals that do |
| | | not completely disappear upon return to room |
| | | temperature |
| B | Clear | amorphous type particles on the surface that |
| | | disappear upon return to room temperature |
| C | Clear | clear with no particles or crystals |

From the above data, it is clear that amount of sodium stearate effects crystallization and amorphous particle forming in the formulation. Thus, in the preferred embodiment, the amount of sodium stearate, according to the present invention, should be greater than 5.0% by weight.

### Example 2

The effect of sodium bicarbonate level was studied and the results are summarized in Tables 3 and 4, below. The compositions, which are set forth in Table 3, were prepared by mixing deionized water, sodium bicarbonate, and 4 sodium EDTA 50%, adding glycerin and heating to 80-90° C. Subsequently the steareth-100, oleth-2, sodium stearate, and cetyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 3

| | Concentration (wt. %) | | | | | |
|---|---|---|---|---|---|---|
| Constituent | A | B | C | D | E | F |
| Glycerin | 60.0 | 60.0 | 60.0 | 60.0 | 60.0 | 60.0 |
| Deionized water | 31.3 | 30.8 | 30.3 | 30.1 | 29.8 | 28.8 |
| Sodium Bicarbonate | 0.1 | 0.5 | 1.0 | 1.25 | 1.5 | 2.5 |
| Sodium Stearate | 5.5 | 5.5 | 5.5 | 5.5 | 5.5 | 5.5 |
| Steareth-100 | 1.5 | 1.5 | 1.5 | 1.5 | 1.5 | 1.5 |
| Oleth-2 | 0.4 | 0.4 | 0.4 | 0.4 | 0.4 | 0.4 |
| Cetyl Alcohol | 0.1 | 0.1 | 0.1 | 0.1 | 0.1 | 0.1 |
| 4 Sodium EDTA 50% | 0.1 | 0.1 | 0.1 | 0.1 | 0.1 | 0.1 |
| Ethylhexylglycerin | 0.3 | 0.3 | 0.3 | 0.3 | 0.3 | 0.3 |
| Alpha Bisabolol (Natural) | 0.2 | 0.2 | 0.2 | 0.2 | 0.2 | 0.2 |
| Fragrance | 0.6 | 0.6 | 0.6 | 0.6 | 0.6 | 0.6 |

TABLE 4

| | Sodium Bicarbonate | | |
|---|---|---|---|
| Composition | % | Comments | Room Temp at 4 weeks |
| A | 0.1% | opaque | opaque |
| B | 0.5% | hazy | hazy |
| C | 1.0% | clear | clear |
| D | 1.25% | clear | clear |
| E | 1.5% | clear | clear |
| F | 2.5% | clear | hazy |

As seen from the above data, compositions A and B are opaque or hazy initially. Compositions C, D, and E are clear and the clarity of stick is not changed after four weeks, but the clarity of composition F changed after 1-2 weeks storage at room temperature (around 20-25° C.).

Sodium bicarbonate, which is solvated by pure water around 10% by weight at 25° C. and around 8% by weight at 18° C., is not soluble in glycerin. The 2.5% sodium bicarbonate in the composition F is actually 8%-9% in a water solution and is in the solvated limit amount. Therefore if we consider the amount of glycerin, higher than 1.5-2.0% of sodium bicarbonate will be crystallized in the stick and the stick become hazy (clearly some of the water in composition F evaporated thereby changing the ratio of sodium bicarbonate to water, thus precipitating excess sodium bicarbonate).

As there appears to be no difference in clarity between composition C, D and E, a 0.75-2.0% of sodium bicarbonate is recommended as a clarifying agent for the composition according to the present invention.

### Example 3

The effect of alkoxylated alcohol levels was studied and the results are summarized in Tables 5 and 6, below. The compositions, which are set forth in Table 5, were prepared by mixing deionized water and sodium bicarbonate, adding glycerin and heating to 80-90° C. Subsequently the steareth-100, oleth-2, sodium stearate, and cetyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 5

| | Concentration (wt. %) | | | | | |
|---|---|---|---|---|---|---|
| Constituent | A | B | C | D | E | F |
| Glycerin | 55.0 | 55.0 | 55.0 | 55.0 | 55.0 | 55.0 |
| Deionized water | 33.3 | 32.7 | 32.3 | 35.2 | 33.7 | 32.7 |
| Sodium bicarbonate | 2.0 | 2.0 | 2.0 | 2.0 | 2.0 | 2.0 |
| Sodium Stearate | 5.5 | 5.5 | 5.5 | 5.5 | 5.5 | 5.5 |
| Steareth-100 | 1.5 | 2.5 | 2.5 | 0.0 | 1.5 | 1.5 |
| Oleth-2 | 0.4 | 0.0 | 0.4 | 0.0 | 0.0 | 1.0 |
| Cetyl Alcohol | 0.1 | 0.1 | 0.1 | 0.1 | 0.1 | 0.1 |
| Ethylhexylglycerin | 0.3 | 0.3 | 0.3 | 0.3 | 0.3 | 0.3 |
| Alpha Bisabolol (Natural) | 0.2 | 0.2 | 0.2 | 0.2 | 0.2 | 0.2 |
| Fragrance | q.s. | q.s. | q.s. | q.s. | q.s. | q.s. |

TABLE 6

| Composition | Comments | Room Temp at 12 Weeks |
|---|---|---|
| A | clear | clear and no crystals |
| B | hazy | hazy and amorphous type particles on the |
| | | surface |
| C | clear | clear but a lot of amorphous type particles |
| D | opaque | inside of stick opaque |
| E | hazy | hazy |
| F | hazy | hazy and a lot of amorphous type inside of |
| | | stick |

With 55% by weight glycerin, around 5.5% by weight sodium stearate, and around 0.3% by weight ethylhexylglycerin, addition of oleth-2 increased the clarity of formulations containing steareth-100. At all concentrations of oleth-2, more than 2.5% by weight of steareth-100 produced amorphous type particles in the formulation. Further, formulations containing over 0.5% by weight oleth-2 were hazy, very soft, and had stability problems. Therefore 1.5-2.0% by weight of a high HLB non-ionic surfactant, such as steareth-100 and 0.0-0.5% by weight of a low HLB non-ionic surfactant, such as oleth-2, with total amount of non-ionic surfactants should be in the range of 1.5-2.5% by weight. Steareth-100, steareth-20, steareth-23, ceteareth-20, oleth-20, oleth-3, oleth-5, laureth-4, isostearate-2, and combinations thereof, have similar effect.

At the low fatty alcohol and glycerin present in the present invention, low HLB non-ionic surfactants reduce unwanted crystallization (crystals of sodium stearate or amorphous type particles of fragrance or alpha Bisabolol and the like) at low temperatures, and increase the ultimate clarity of the formulations.

### Example 4

The effect of fatty alcohols was studied and the results are summarized in Tables 7 and 8, below. The compositions, which are set forth in Table 7, were prepared by mixing deionized water and sodium bicarbonate, adding glycerin and heating to 80-90° C. Subsequently the steareth-100, sodium stearate, and stearyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 7

| | Concentration (wt. %) | | | | |
|---|---|---|---|---|---|
| Constituent | A | B | C | D | E |
| Glycerin | 60.00 | 60.0 | 60.00 | 60.0 | 60.00 |
| Deionized water | 31.15 | 30.95 | 30.70 | 30.45 | 30.20 |
| Sodium Bicarbonate | 0.90 | 0.90 | 0.90 | 0.90 | 0.90 |
| Sodium Stearate | 5.00 | 5.00 | 5.00 | 5.00 | 5.00 |
| Steareth-100 | 1.50 | 1.50 | 1.50 | 1.50 | 1.50 |
| Stearyl Alcohol | 0.00 | 0.25 | 0.50 | 0.75 | 1.00 |
| Ethylhexylglycerin | 0.60 | 0.60 | 0.60 | 0.60 | 0.60 |
| Alpha Bisabolol (Natural) | 0.20 | 0.20 | 0.20 | 0.20 | 0.20 |
| Fragrance | q.s. | q.s. | q.s. | q.s. | q.s. |

TABLE 8

| Composition | Comments |
|---|---|
| A, B, and C | clear |
| D | hazy and softer than composition A, B, and C |
| E | opaque and very soft |

And higher than 0.5% fatty alcohol, stick become hazy and too soft to use as deodorant stick. The initial clarity of composition A appears better than compositions B and C, but at low temperature, composition A becomes very hazy. Therefore a fatty alcohol concentration in the range of 0.2-0.5% by weight is preferred.

### Example 5

The effect of low HLB non-ionic surfactant with high amount of fatty alcohol was studied and the results are summarized in Tables 9 and 10, below. The compositions, which are set forth in Table 9, were prepared by mixing deionized water and sodium bicarbonate, adding glycerin and heating to 80-90° C. Subsequently the steareth-100, oleth-5, sodium stearate, and stearyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 9

Concentration (wt. %)

| Constituent | A | B |
|---|---|---|
| Glycerin | 60.00 | 60.00 |
| Deionized water | 31.15 | 30.95 |
| Sodium bicarbonate | 1.25 | 1.25 |
| Sodium Stearate | 5.50 | 5.50 |
| Steareth-100 | 1.50 | 1.50 |
| Oleth-5 | 0.00 | 0.20 |
| Stearyl Alcohol | 0.50 | 0.50 |
| Ethylhexylglycerin | 0.60 | 0.60 |
| Alpha Bisabolol (Natural) | 0.20 | 0.20 |
| Fragrance | q.s. | q.s. |

TABLE 10

| Composition | Comments | 4° C. at 8 weeks |
|---|---|---|
| A | clear | clear and no crystals |
| B | clear | clear but crystals and amorphous type particles |

At 0.5% stearyl alcohol, addition of oleth-5 has negative effect for low temperature stability of stick.

### Example 6

The effect of ethylhexylglycerin was studied and the results are summarized in Tables 11 and 12, below. The compositions, which are set forth in Table 11, were prepared by mixing deionized water and sodium bicarbonate, adding glycerin and heating to 80-90° C. Subsequently the steareth-100, sodium stearate, and stearyl alcohol were added and the mixture cooled. After cooling to about 67-70° C., fragrance and the deodorant active materials, ethylhexylglycerin, and alpha Bisabolol, were added. The final compositions were then poured into containers and cooled to room temperature.

TABLE 11

| | Concentration (wt. %) | | |
|---|---|---|---|
| Constituent | A | B | C |
| Glycerin | 60.00 | 60.00 | 60.00 |
| Deionized water | 31.15 | 30.95 | 30.70 |
| Sodium bicarbonate | 0.90 | 0.90 | 0.90 |
| Sodium Stearate | 5.00 | 5.00 | 5.00 |
| Steareth-100 | 1.50 | 1.50 | 1.50 |
| Stearyl Alcohol | 0.25 | 0.25 | 0.25 |
| Ethylhexylglycerin | 0.00 | 0.30 | 0.60 |
| Alpha Bisabolol (Natural) | 0.20 | 0.20 | 0.20 |
| Fragrance | q.s. | q.s. | q.s. |

TABLE 12

| Composition | Comments | 4° C. at 8 weeks |
|---|---|---|
| A | clear | clear and no crystals |
| B | clearer | clear and no crystals |
| C | clearest | clear and no crystals |

The clarity of composition C is better than composition B and the clarity of composition B is better than composition A. Therefore ethylhexylglycerin, which is used in this invention as a deodorant active ingredient, also increases the clarity of the formulations. Further, addition of ethylhexylglycerin does not appear to affect any other properties, such as hardness. However, at concentrations higher than 0.6% by weight, the clarity of stick is less that other compositions.

48 Hour Human Patch Test

### Formula

Following formula was used for this test

TABLE 13

Concentration (wt. %)

| Constituent | A | B | C | D |
|---|---|---|---|---|
| Glycerin | — | — | 60.00 | 60.00 |
| Propylene glycol | — | 70.00 | — | — |
| Deionized water | 100.00 | 21.80 | 29.35 | 30.35 |
| Sodium Bicarbonate | — | — | 0.95 | 0.95 |
| Sodium Stearate | — | 8.00 | 5.50 | 5.50 |
| Steareth-100 | — | — | 1.50 | 1.50 |
| Stearyl Alcohol | — | — | 0.50 | 0.50 |
| Ethylhexylglycerin | — | — | 0.60 | 0.60 |
| Alpha Bisabolol (Natural) | — | — | 0.20 | 0.20 |
| Olive water | — | — | 1.00 | — |
| Triclosan | — | 0.20 | — | — |
| Fragrance | — | q.s. | q.s. | q.s. |

The above four different compositions were tested. 19 subjects were used in this test.

Four drops of each of the above formulations were placed on separate gauzes and attached to the subject's volar forearm using a strong occlusive medical tape. After 48 hours, the sample containing gauze was removed and the test sites were ranked according to the degree of irritation. The results are tabulated in Table 14 below. Ranking was done using the following: 0=No evidence of any reaction; 1=Minimal, faint uniform or spotty erythema; 2=Pink uniform erythema covering most of the patch site; 3=Pinkish-red erythema covering most of the patch site; 4=Bright red erythema; and 5=Deep red erythema.

TABLE 14

| Subject | A | B | C | D |
|---|---|---|---|---|
| F | 1 | 2 | 0 | 1 |
| F | 2 | 3 | 1 | 0 |
| F | 0 | 0 | 1 | 0 |
| F | 0 | 0 | 1 | 0 |
| F | 0 | 2 | 0 | 0 |
| F | 1 | 2 | 1 | 0 |
| F | 1 | 2 | 0 | 1 |
| F | 1 | 1 | 0 | 0 |
| F | 1 | 3 | 0 | N/A |
| F | 0 | 3 | 0 | 0 |
| M | 0 | 3 | 2 | 0 |
| M | 0 | 2 | 2 | 0 |
| M | 0 | 1 | 1 | 1 |
| M | 1 | 3 | 0 | 0 |
| M | 0 | 3 | 0 | 0 |
| M | 1 | 2 | 0 | 0 |
| M | 0 | 3 | 0 | 0 |
| M | 1 | 2 | 0 | 0 |
| M | 1 | 3 | 0 | 0 |
| Mean | 0.58 | 2.11 | 0.47 | 0.17 |
| Standard Dev | 0.61 | 0.99 | 0.70 | 0.38 |

The mean response to deionized water (0.58) was minimal. The skin irritation of the two formulations according to the present invention (C and D) above appears to be similar to or less than that of deionized water. The skin irritation of the sample that includes propylene glycol and triclosan (B, 2.11), however, is very strong showing a pink uniform erythema covering most of the patch site. Thus, the formulations that are essentially free of propylene glycol, according to the present invention demonstrate a clear advantage over those containing propylene glycol.

The antimicrobially synergistically effect of alpha Bisabolol (Natural) for glyceryl monoalkyl ether was studied and the results are summarized in Tables 15 and 16, below. The compositions, which are set forth in Table 15, were prepared by mixing deionized water and sodium bicarbonate, adding glycerin and heating to 80-90° C. Subsequently the steareth-100, sodium stearate, and stearyl alcohol were added and the mixture cooled. After cooling to about 67-70° C., and the deodorant active materials, ethylhexylglycerin, and alpha Bisabolol, were added. The final compositions were then poured into containers and cooled to room temperature.

To inoculate test microorganisms to the formula directly, the amount of sodium stearate was reduced to 1.00% from 5.00-10.00%. Therefore following formula is gel type.

TABLE 15

| | Concentration (Wt. %) | | | | | |
|---|---|---|---|---|---|---|
| Constituent | A | B | C | D | E | F |
| Glycerin | 60.00 | 60.00 | 60.00 | 60.00 | 60.00 | 60.00 |
| Deionized water | make | make | make to | make to | make | make to |
| | to 100 | to 100 | 100 | 100 | to 100 | 100 |
| Sodium bicarbonate | 0.95 | 0.95 | 0.95 | 0.95 | 0.95 | 0.95 |
| Sodium Stearate | 1.00 | 1.00 | 1.00 | 1.00 | 1.00 | 1.00 |
| Steareth-100 | 1.50 | 1.50 | 1.50 | 1.50 | 1.50 | 1.50 |
| Stearyl Alcohol | 0.50 | 0.50 | 0.50 | 0.50 | 0.50 | 0.50 |
| Ethylhexylglycerin | 0.60 | 0.60 | 0.00 | 0.00 | 0.30 | 0.30 |
| Alpha Bisabolol (Natural) | 0.20 | 0.00 | 0.20 | 0.00 | 0.10 | 0.00 |

Inoculate test microorganisms to provide a suspension containing from 105 to 106 microorganisms/ml and after 1 hr, 5 hr and 24 hr, check the surviving microorganisms by incubating in the soybean-casein digest agar for 24 hours at 32.5° C. the results are summarized below.

TABLE 16

| | Sample | | | | | |
|---|---|---|---|---|---|---|
| | A | B | C | D | E | F |
| Initial microorganism | 2.3 * 10⁶ | 2.3 * 10⁶ | 2.3 * 10⁶ | 2.3 * 10⁶ | 2.1 * 10⁵ | 2.1 * 10⁵ |
| Concentration (CFU/ml) | | | | | | |
| 1 hr recovery | 6.8 * 10³ | 5.9 * 10⁵ | 2.4 * 10⁵ | 2.6 * 10⁵ | 1.2 * 10⁵ | 2.0 * 10⁵ |
| surviving microorganism | | | | | | |
| concentration (CFU/ml) | | | | | | |
| 5 hours recovery | 12.5 | 1.3 * 10⁴ | 3.6 * 10⁵ | 2.6 * 10⁵ | — | — |
| surviving microorganism | | | | | | |
| concentration (CFU/ml) | | | | | | |
| 24 hours recovery | 0.0 | 60.0 | 4.9 * 10⁴ | 3.2 * 10⁵ | 0.0 | 9.8 * 10⁵ |
| surviving microorganism | | | | | | |
| concentration (CFU/ml) | | | | | | |

Sample A, which includes alpha Bisabolol (Natural) at 0.2% by weight and ethylhexylglycerin at 0.6% by weight, exhibit a 3.0 log reduction after 1 hour, a 5.0 log reduction after 5 hours, and no viability after 24 hours for the Staphylococcus. Sample B, which includes only ethylhexylglycerin at 0.6% by weight, exhibits a 1.0 log reduction for 1 hour, 2.0 log reduction after 5 hours, and 5.0 log reduction after 24 hours. There appears to be no anti-microbial activity of alpha Bisabolol (Natural) at 0.2% by weight (with no ethylhexylglycerin). However, alpha Bisabolol (Natural), which lacks anti-microbial activity by itself, exhibited a surprising and unexpected synergistic effect on the anti-microbial activity of ethylhexylglycerin, thereby greatly increasing the anti-microbial activity of the ethylhexylglycerin.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

1. A clear deodorant composition comprising glycerin as a gelling agent, water as a solvent, a clarifying effective amount of a bicarbonate compound as a clarifying agent and a deodorant active material.

2. The clear deodorant composition according to claim 1 wherein the deodorant active material is a mixture comprising at least one glyceryl monoalkyl ether and an anti-microbially synergistically effective amount of at least one monocyclic, unsaturated sesquiterpene alcohol.

3. The clear deodorant composition according to claim 2 wherein the at least one glyceryl monoalkyl ether is ethylhexylglycerin.

4. The clear deodorant composition according to claim 2 wherein the at least one monocyclic, unsaturated sesquiterpene alcohol is alpha-Bisabolol.

5. The clear deodorant composition according to claim 3 wherein the at least one monocyclic, unsaturated sesquiterpene alcohol is alpha-Bisabolol.

6. The clear deodorant composition according to claim 3 wherein the at least one bicarbonate compound is sodium bicarbonate.

7. The clear deodorant composition according to claim 4 wherein the at least one bicarbonate compound is sodium bicarbonate.

8. The clear deodorant composition according to claim 5 wherein the at least one bicarbonate compound is sodium bicarbonate.
